(19) United States
(12) Patent Application Publication
(10) Pub. No.: US 2023/0245739 A1
(43) Pub. Date: Aug. 3, 2023

(54) Patient Cluster Identification System and Method

(71) Applicant: Parkland Center for Clinical Innovation, Dallas, TX (US)

(72) Inventors: Yusuf Tamer, Dallas, TX (US); Albert Karam, Addison, TX (US); Steve Miff, Dallas, TX (US); Brett Andrew Moran, Dallas, TX (US); Joseph Longo, Southlake, TX (US)

(21) Appl. No.: 18/098,099

(22) Filed: Jan. 17, 2023

Related U.S. Application Data
(60) Provisional application No. 63/305,252, filed on Jan. 31, 2022.

(51) Int. Cl. G16H 20/00; G16H 40/20; G16H 50/70

(52) U.S. Cl. CPC G16H 20/00; G16H 40/20; G16H 50/70

(57) ABSTRACT

What is disclosed is a computerized system and method that include a data ingestion logic module that automatically receive, cleanse, and process data associated with a plurality of patients to create a plurality of holistic patient records that include identity and demographic metrics associated with each patient, clinical metrics associated with each patient, clinical utilization metrics associated with each patient, social determinants of health metrics associated with each patient at the blockgroup level, and social determinants of health metrics associated with each patient at the census track level. The system and method is configured to identify a plurality of cluster-defining metrics that include demographics metrics, insurance coverage metrics, healthcare utilization metrics, and social determinants of health metrics, and automatically identify a plurality of patient clusters based on the cluster-defining metrics, assign each patient to an identified patient cluster, and generate a recommendation for holistic and targeted care plans and programs.

### RELATED APPLICATION

This patent application claims the benefit of U.S. Provisional Application No. 63/305,252 filed on Jan. 31, 2022, which is incorporated herein by reference in its entirety.

### FIELD

The present disclosure relates to a system and method that analyzes patients' clinical and non-clinical data and assigns each patient to a predetermined cluster based on a set of data features to provide insight that contribute to improved workflows, targeted programs, and healthcare related policies.

### BACKGROUND

Many people in the U.S. do not have access to adequate timely healthcare. Traditional disease-based clinical programs have been effective in managing and treating specific medical conditions, but often fail to holistically treat the whole person. Conventional healthcare systems lack profound insight into the patient population's patterns of access to healthcare and do not take into account the patients' complex lifestyle factors, especially with respect to barriers to healthcare access, e.g., social vulnerabilities, transportation barriers, language barriers, and lack of insurance coverage.

### BRIEF DESCRIPTION OF THE DRAWINGS

FIG. 1 is a flowchart of an embodiment of a patient cluster identification system and method according to the teachings of the present disclosure;

FIG. 2 is an example GUI map plot of the geographical distribution and characteristics (gender, age, marital status, race, chronic conditions, and insurance coverage) of the patient population analyzed by the system and method of the teachings of the present disclosure;

FIG. 3 is a graphical illustration of the patient clusters created by the patient clustering system and method of the present teachings providing insight to the in-person visit count and emergency department visits of the patient clusters;

FIG. 4 are example GUI heatmap plots of geographical distribution of eight patients cluster classifications of a given patient population according to the teachings of the present disclosure;

FIGS. 5-8 are example GUI heatmap plots of geographical distribution and inherent and descriptive characteristics of G1, G2, G5, and G8 patients clusters according to the teachings of the present disclosure;

FIG. 9 is a graphical illustration of a recommendation of an integrated practice unit targeting the cardio-metabolic high risk patient clusters G1 and G8 according to the teachings of the present disclosure;

FIG. 10 are simplified flow diagrams of an embodiment of the patient cluster system and method according to the teachings of the present disclosure;

FIG. 11 is a simplified block diagram of the exemplary operating environment of the patient cluster system and method; and

FIG. 12 is a table of exemplary metrics used for patient cluster identification according to the teachings of the present disclosure.

### DETAILED DESCRIPTION

Many people in the U.S. do not have access to adequate healthcare. Having insight into patient's patterns of access to healthcare can help to remove impediments that patients experience when seeking medical attention for their physical and mental health. Although traditional disease-based programs have been effective, they often fail to holistically treat and care for the whole person, align and address concurrently the complex needs of patients, and facilitate stronger provider-to-patient and patient-to-patient connections and support. Instead of grouping patients by their primary disease/diagnosis (e.g., diabetes, hypertension, etc.), grouping and patients into cohorts with others who have high degrees of common clinical, personal, and behavioral characteristics can better facilitate the creation and deployment of new programs such as integrated practice units, deployment of targeted digital programs, virtual support groups, and targeted outreach and communication. The resulting programs and treatment plans can more readily incorporate a wide variety of patient-centered, whole-person approaches and workflows.

The patient cluster identification system and method described herein provide a novel approach for patient segmentation and clustering using methods such as machine learning to develop holistic, patient-centered programs and treatment plans. The patient cluster identification system and method described herein are operable to identify patient clusters based on a given set of cluster-defining data features (also referred to as metrics or factors) and a given set of cluster-descriptive data features, assign a particular patient to an identified patient cluster, and generate one or more recommendations, such as a care plan for the particular patient, targeted programs for one or more patient clusters, and improved workflows. The patient clusters also provide insight to the patients and how they access healthcare so that more effective outreach programs and services may be designed and implemented to improve access to healthcare.

As used herein, the term “cluster” refers to a group of individuals with similar patterns of health care utilization and access. Clusters are created by an unsupervised machine learning method called clustering. Clusters are not just physical or geographic groupings. The patient clusters are created using multiple factors or metrics. Demographic background, clinical interaction and utilization, and social determinants of their neighborhoods are confounding factors for the clusters. The term “clustering” refers to a method of unsupervised machine learning used by the patient cluster identification system and method described herein. The preferred clustering algorithm is deterministic, hence, repeatable. In other words, patients with similar healthcare access and utilization patterns will cluster together consistently. The patient clustering logic may be run periodically (e.g., hourly, daily, weekly, bi-weekly, monthly, quarterly, bi-annually, or annually) or dynamically to analyze real-time data as they become available and accessible.

Optimal access to healthcare is defined as getting timely personal health services that results in the best possible health outcomes. This definition focuses on three aspects of access: (1) time to the care facility, (2) individual and community characteristics, and 3) health insurance coverage. The first factor may depend on the amount of time to the care facility, which may include transit time, availability of public transit, the number and distribution of care facilities in a geographical area served by those facilities. Socioeconomically vulnerable populations (including those in rural or peripheral areas) often fail to receive proper frequency of care due to longer travel times (and costs) to adequately equipped health care facilities (which often cluster around densely populated urban areas) and face challenges to use public transport and motorized vehicles. The second factor means accessibility to patients with specific characteristics of demographics, Social Determinants of Health (SDoH), types of clinics visited, utilization of the emergency facilities, inpatient and outpatient resources, and length of stay (LOS). For example, disparities in accessing healthcare facilities for some ethnic and racial communities are well documented. Other factors such as language, education, economic stability, and social and community context also affect access to healthcare. Combining personal characteristics (e.g., age, gender, race) and social determinants (e.g., family structure, transportation, income) with utilization of clinics, emergency departments, and inpatient resources can provide a wider lens to understand access barriers. The third factor relates to whether the patient has health insurance coverage that would cover visits to care facilities. Health insurance is a crucial component for healthcare access and improved outcomes. For example, insured (non-elderly) adults and children have a higher likelihood of receiving proper frequency visits than their uninsured counterparts.

Some of the questions that are answered by the patient clustering logic include: what are the characteristics of heavy emergency facility users? What are the compounding factors for utilization profiles? What can be done to minimize emergency to outpatient visit ratios? What are the SDoH features that impact access to specific healthcare facilities?

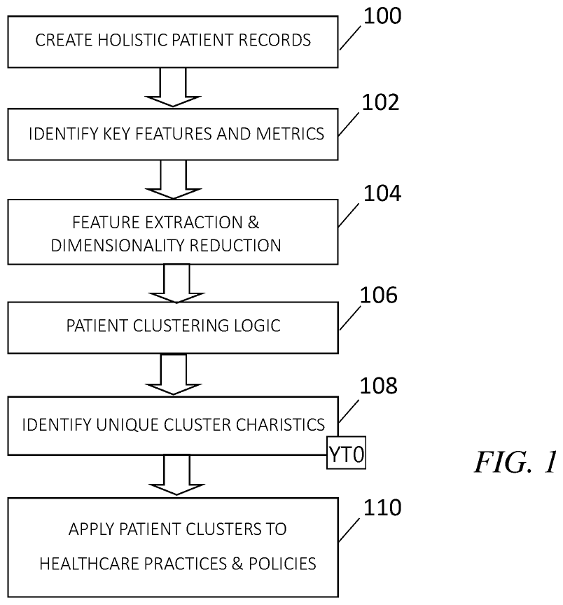

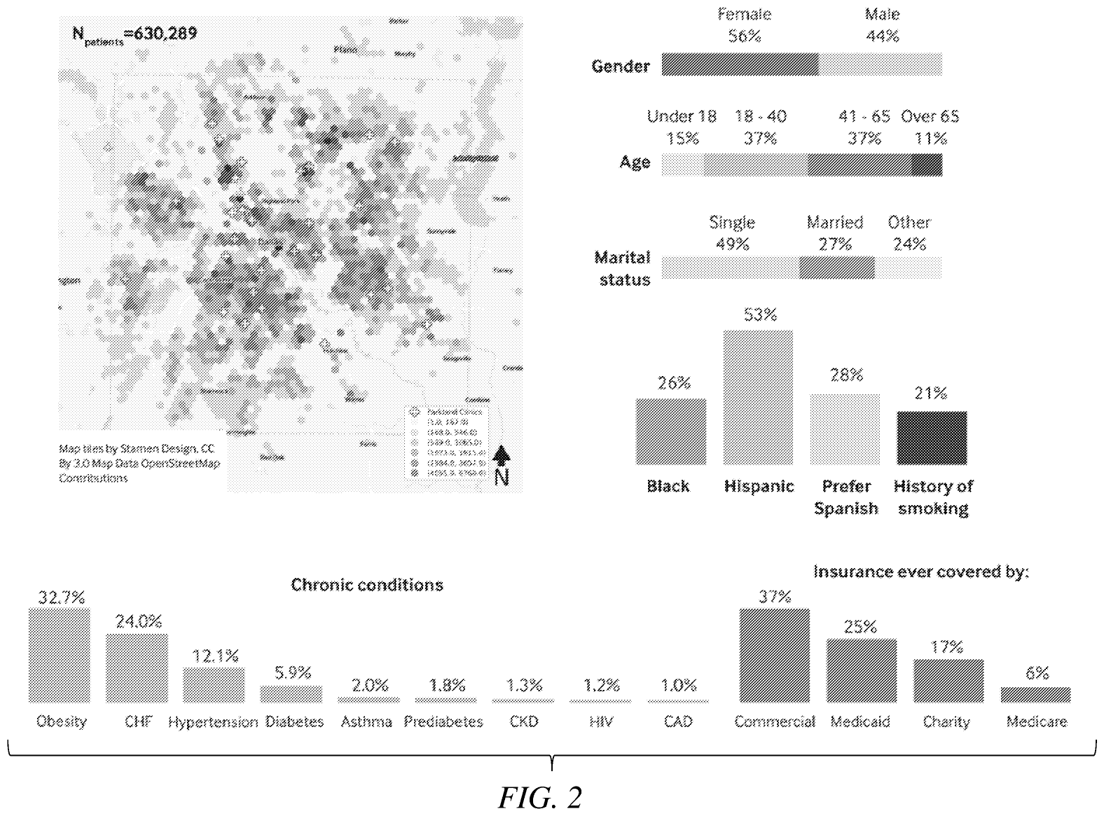

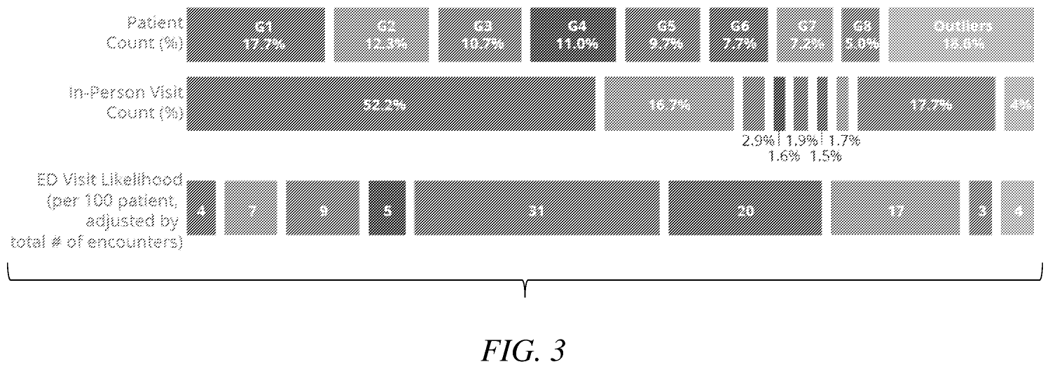

As shown in FIG. 1, the first step 100 of the method is to create a holistic patient record that is clean and complete. This means that the data record includes metrics and data types that are well-defined, and missing values are imputed, with duplicate data identified and removed. The record includes metrics beyond patient-specific clinical information, such as SDoH. Data identification, ingestion, integration, and staging are carried out. This process includes deciding which data elements to use, cleaning/integrating data, enriching the data from geo level to patient level (their zip code affects their health in subtle/less subtle ways). Clinical and non-clinical data, which include an aggregation of the patients' data over a period of time and real-time data that are collected about the patients' visits at one or more healthcare institutions, for example, care facilities that are part of Parkland Health (PH) that serve residents that live in Dallas County and surrounding areas. In a sample, data associated with 24 million encounters over a period of six years were collected from one million patients who have valid addresses in the Dallas-Fort Worth area. The collected data include, e.g., 129 features specific to each patient, including unique identifiers, birthdate, death date (if applicable), domicile address, other location information (e.g., block group, census tract, latitude, longitude, and genocode), demographics (ethnicity, race, age), preferred language, marital status, tobacco use, number and type of encounters, insurance coverage, utilization, disease registry status, Charlson Comorbidity index, etc. Additionally, data for, e.g., 171 features, are collected from the patients' neighborhoods (e.g., census block, block groups, census tracts), such as prevalence of chronic diseases, employment, income level, food insecurity, education level, population density, distance or accessibility to facility locations, etc. The metrics also includes data elements that provide an understanding of transportation-related access barriers such as transit times to the closest outpatient clinic/hospital and proximity to public transportation. Therefore in an embodiment of the present system and method, the initial number of metrics associated with each patient is 300. The domicile locations of these patients, when plotted on a heatmap, are shown in FIG. 2.

FIG. 2 shows a plot of these patients' domicile with the location of Parkland Health care facility locations and a further breakdown of the patients by gender (56% female compared to 44% male), race/ethnicity (26% black and 53% Hispanic), age group (15% under 18, 37% between 18 and 40, 37% between 41 and 65, and 11% over 65), language preference (28% prefer Spanish), marital status (49% single compared to 27% married), and tobacco use (21% have a history of tobacco use). The data also yielded cluster-defining characteristics about these patients—almost 40% had more than three encounters per year, and 26% had more than six encounters per year. Among the patients, 14% had more than one emergency room visit, 37% had more than one outpatient visit, and 20% had more than three outpatient visits. 76% of the patients visited more than two clinic locations. The average time since the last encounter is six months. The data also provided descriptive characteristics about these patients, including clinical complexity, disease registries (asthma, chronic kidney disease, hypertension, diabetes, HIV), cost per patient per year, cost per visit, and Covid vaccination status.

The step 102 of identifying key features/metrics to be used for clustering may start with an initial number of metrics, e.g., 300. These initial metrics describe, for example, the patients' name, birthdate, age, gender, address, blockgroup, census tract, latitude, longitude, county, ethnicity, race, language, smoking data, marital status, first encounter, last encounter, payor information, clinical encounter information, disease registry information, disease diagnoses, and vaccination information. The initial metrics also include SDoH data based on the census tract and blockgroup of the patient, including, for example, disease prevalence information, smoking prevalence, dental visit prevalence, mammography screen prevalence, sleep pattern prevalence, economic status prevalence, employment prevalence, household makeup, vehicle ownership, language proficiency, paycheck predictability, food insecurity, household structure, transportation mode, education level, distance to Parkland Health facilities, Internet access, etc.

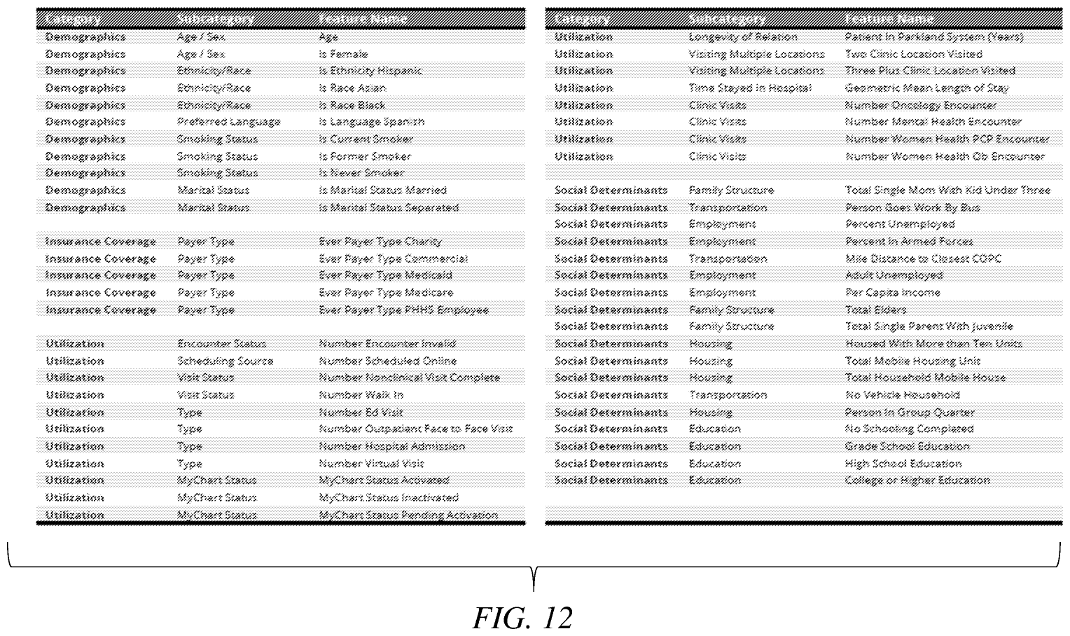

In step 104, feature extraction and reduction is applied in two ways. First, metrics related to medical diagnosis are removed so that clustering is not impacted by any medical diagnosis directly. A purposeful decision was made not to use medical diagnosis into the clustering but are used to describe the key features and insights once the clusters are identified. In this way, patients are not treated as diseases but as whole people. Second, multicollinearity relationships between metrics are evaluated and highly multicollinear metrics (for example, neighborhood unemployment rate versus prevalence of uninsured rate) are removed to decrease information redundancy. Variation Inflation Factors (VIF) may be used to decrease the information redundancy in the collected dataset. VIF measures the multicollinearity of the metrics. Due to the highly interwoven nature of social determinant features, metrics may be eliminated without losing insight. For example, socio-economically affluent neighborhoods have higher average median income, median home value, and more green spaces/park areas. So, there is shared information between these metrics that can be preserved at high percentages by utilizing only a small portion of them. This feature reduction process removes the descriptor metrics from the initial metrics and results in a smaller number of defining metrics that are used for forming the patient clusters. In this step, the number of metrics is reduced from an initial number of, e.g., 300, to, e.g., 53. These 53 metrics are the cluster defining metrics, and the remaining 247 are the cluster descriptor metrics. The 53 cluster-defining metrics are used to specify two or more (e.g., eight) patient clusters and the 247 cluster-descriptive metrics are used to describe these patient clusters. FIG. 12 is a table of exemplary metrics used to define the clusters, i.e., the cluster-defining metrics/features.

Thereafter in step 106, a dimensionality reduction technique such as Uniform Manifold Approximation and Projection (UMAP) is used to reduce the dimensionality (e.g., 53 dimensions projected to 2 dimensions) to improve data visualization and interpretation. UMAP preserves the similarities between patients and provides a transformer where the same feature reduction can be applied to new patients or to patients with new updated information. Next, HDBScan, a deterministic, hierarchical, clustering algorithm is used to form the eight patient clusters. The cluster-defining metrics include variables that provide insights to the patients' demographics, their utilization of healthcare facilities, and SDoH data of their neighborhoods. The cluster-descriptive metrics include, e.g., specialty clinic visits, home visits, chronic disease presence, Charlson Comorbidity index, Community Vulnerability Compass Index, etc. All medical diagnosis data are then used along with the defining metrics and the descriptor metrics to perform descriptive analyses to understand the cluster profiles. The use of both UMAP and HDBScan algorithms enables the assignment of a patient into a cluster based on the patient's demographics, SDoH, and their initial interaction with PH.

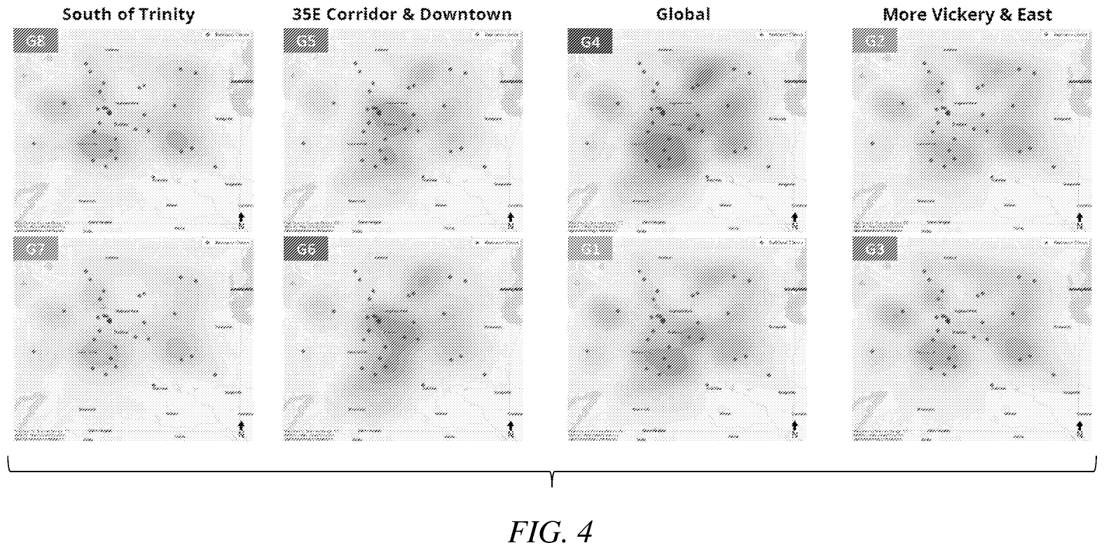

In steps 108 and 110, after clustering, all medical diagnosis data are reintroduced and both sets of the defining metrics and descriptor metrics are used to perform descriptive analyses to thoroughly understand the cluster profiles. The patient clusters are mapped to more clearly identify and visualize neighborhoods of focus, clinic utilization, and travel times to the closest facilities. FIG. 4 show heatmaps that show the geographical distribution of the patients in each of the eight clusters, where some of the groups share similarities in their population distribution.

The patient clustering step may employ unsupervised, machine learning, and/or artificial intelligence methods. The algorithm determines the clusters—patients are not pre-assigned, but after clustering, then analysis of what is meaningful in each cluster. A non-sequential/holistic approach is used as to entire persons. The cluster defining step (i.e., step 104) and patient clustering step (i.e., step 106) may be performed on a periodic basis, or be dynamically performed to take into account of real-time data to analyze data related to new patients and new data of existing patients.

In step 108, the unique characteristics of each cluster may be identified by using descriptive analytics to describe each cluster such as location density; demographics; access patterns (crosses); cost of care; medical complexity; engagement (vaccination; virtual vs. in-person access); medical complexity; “life complexity and support”; SDoH needs; barriers to access; etc.

The final step 110 applies the identified clusters to enhance access and quality of care for the patients, which may include integrated practice unit design, new healthcare sites, digital front door for telehealth engagement, integration into medical records at point of care, virtual support groups, and payer (claim-based) clustering. In step 110, the patient cluster identification system and method may use insights gained from this analysis to recommend, for example: 1. Classify patients as members of specific clusters so that patient needs and care are better anticipated and planned. 2. Situate and operate care facilities to better serve certain patient populations based on utilization pattern of clusters. 3. Inform and cooperate with county and city transportation agencies to improve access to care facilities. 4. Design and implement more effective patient outreach programs, such as targeting patients in specific clusters that are more likely to use specific services.

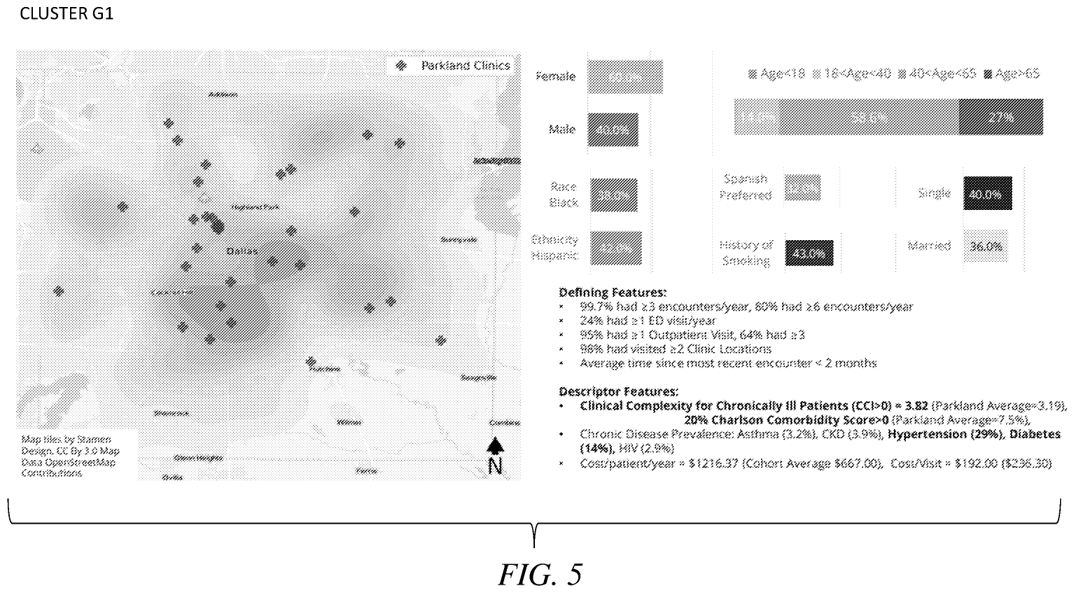

As shown in FIG. 5, the patient population size of the eight patient clusters (G1-G8) varies from 5% to 17.7% of the total patient population. Patients that cannot be included in a defined cluster are assigned to an outliers cluster, which includes 18.6% of the total patient population. Some insights may be gathered from these clusters. Patients in the G1 cluster, the largest cluster, account for 53.7% of all clinical encounters, while patients in the G8 cluster have, at 20%, more encounters per patient than all other clusters. Patients in the G2 cluster prefer in-person (defined to include outpatient face-to-face visits, emergency department visits, and hospital admissions) visits while those in the G8 cluster tend to utilize other forms of encounters (e.g., telehealth and phone consultations). Patients in cluster G5 are ten times more likely to use the emergency room services than other patients.

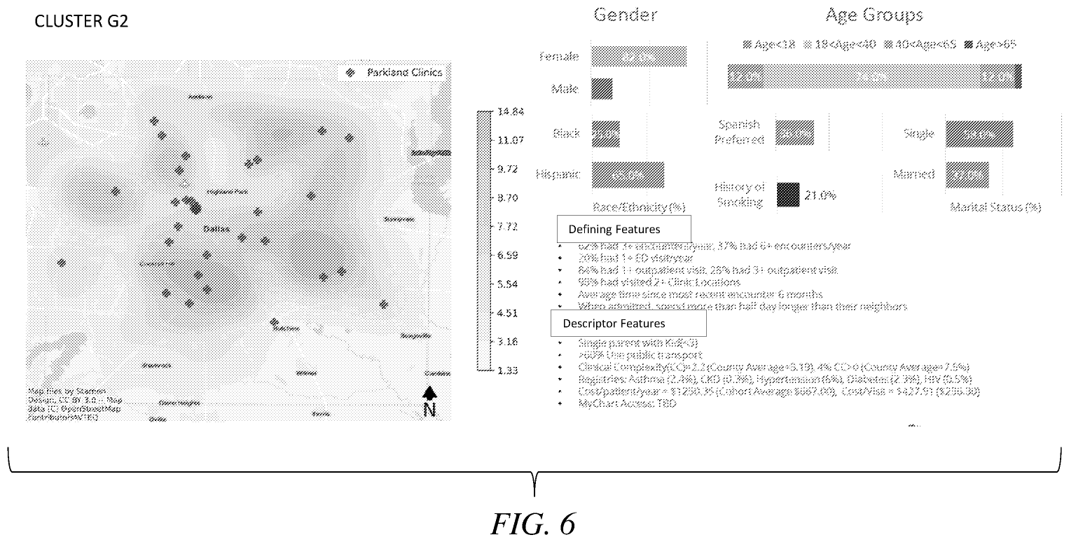

Referring to FIG. 6, the G1 cluster is the largest cluster by population and had more encounters than other groups. The cluster-defining features include, for example: 99.7% of the patients in this cluster had more than three encounters per year and 80% of the patients in this cluster had more than six encounters per year. 24% of these patients had greater than one emergency department visit per year, and 95% had greater than one outpatient visit, while 64% had greater than three outpatient visits. 98% of the patients in this cluster visited two or more clinic locations in the year. The cluster-descriptive features of this group include: clinical complexity for clinically ill patients in this group is 3.82 compared to the county average of 3.19. Further, 20% of patients in this cluster have a Charlson comorbidity score greater than zero, compared to the Parkland Health average of 7.5%. Further, the patients in this cluster have chronic diseases: 29% have hypertension, 14% have diabetes, 3.2% have asthma, 3.9% have chronic kidney disease, and 2.9% have HIV. For this group of patients, the cost per patient per year is approximately $1216.37 compared to the cohort average of $667, where the cost per visit is $192 compared to the cohort average of $236.

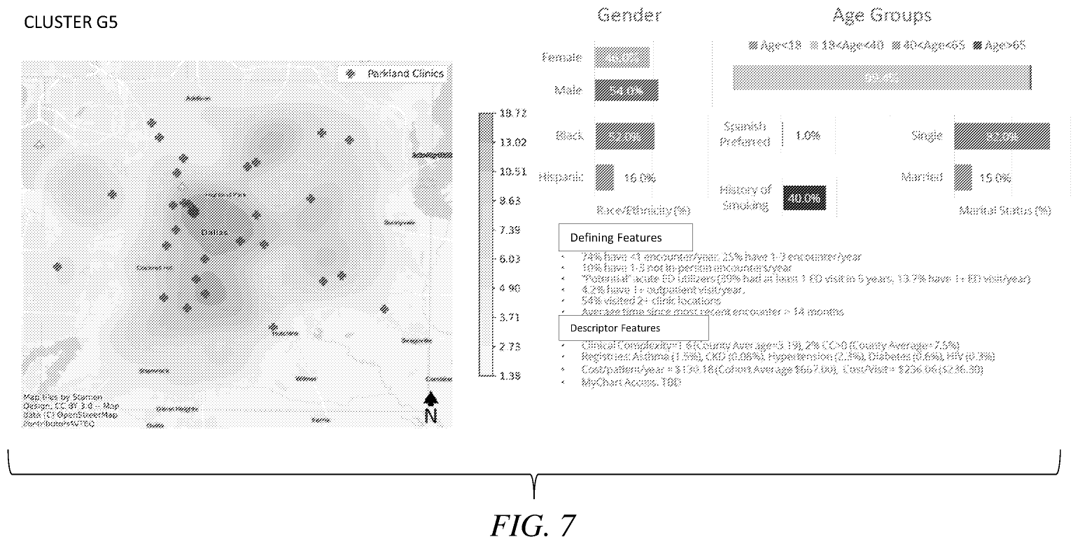

Referring to FIG. 7, using cluster G2 as an example, 62% of the patients in this cluster had more than three encounters per year and 37% of them had more than six encounters per year. 20% of the patients had more than one emergency department visit per year, 84% had more than one outpatient visit, and 28% had more than three outpatient visits. A majority, 98%, of the patients in this group had visited more than two clinic locations per year. Among the cluster-descriptive features are: patients in this cluster tend to be single parents with children under three years old. A majority of them (60%) use public transportation to access the healthcare facilities. The clinical complexity for patients in this group is 2.2 compared to the county average of 3.19. Further, the patients in this cluster have chronic diseases: 6% have hypertension, 2.3% have diabetes, 2.4% have asthma, 0.3% have chronic kidney disease, and 0.5% have HIV. For this group of patients, the cost per patient per year is approximately $1250.35 compared to the cohort average of $667, where the cost per visit is $427.91 compared to the cohort average of $236.

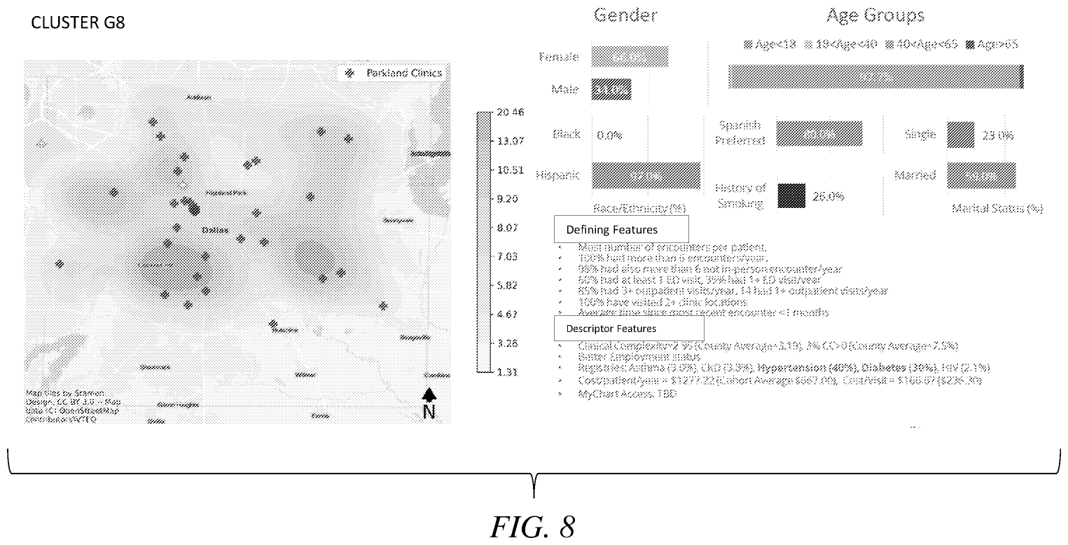

Referring to FIG. 8, the cluster-defining features for cluster G5 are, for example: 74% of the patients in this cluster had fewer than one encounter per year, and 25% have 1-3 encounters per year. 10% of the patients in this group have 1-3 non-in-person encounters per year. This group include potentially acute emergency department utilizers—39% had at least one emergency room visit in five years, and 13.7% had more than one emergency room visit per year. A small percentage, 4.2%, had more than one outpatient visits, and the majority, 54%, visited more than two clinic locations. The average time since the last encounter is 14 months. The cluster-descriptive features include, for example, the clinical complexity of encounters with these patients are 1.6 compared to the county average of 3.19. The cost per patient per year in this cluster is $130.18 compared to the cohort average of $667, and the cost per visit is approximately $236.06 which is close to the cohort average. Further, the chronic disease make-up of this cluster is: 2.3% have hypertension, 0.6% have diabetes, 1.5% have asthma, 0.08% have chronic kidney disease, and 0.3% have HIV.

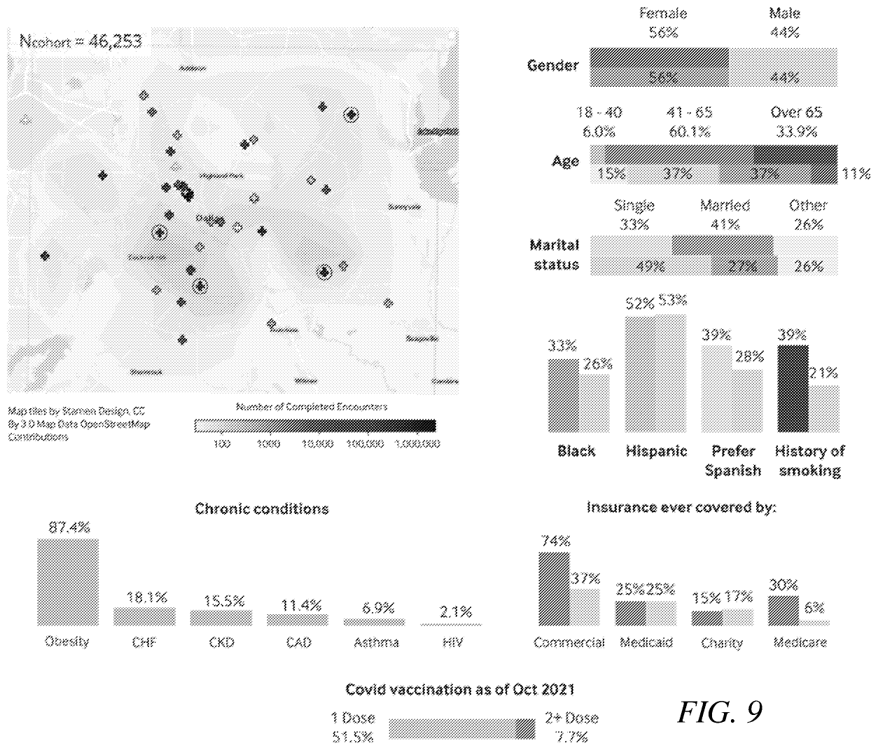

Referring to FIG. 9, the cluster-defining features of cluster G8 include, for example, the patients in this cluster had the most clinical encounters per year, where all of them had more than six encounters per year. A high percentage, 98%, of these patients also had more than six non-in-person visits per year. This group is also high emergency department utilizers—60% of this patient population had at least one emergency department visit and 35% had more than one emergency room encounter per year. 85% of these patients had more than three outpatient visits per year, and 14% had more than one outpatient visits per year. 100% of the patients in this cluster have visited two or more clinic locations. The average time since the last encounter is shorter than one month. The cluster-descriptive features include, for example: the cost per patient per year in this cluster is $1277.22 compared to the cohort average of $667, and the cost per visit is $166.67 compared to the cohort average of $236.30. Clinical complexity for the encounters is 2.95 compared to the county average of 3.19. Further, the chronic disease make-up of this cluster is: 40% have hypertension, 30% have diabetes, 3.0% have asthma, 3.8% have chronic kidney disease, and 2.1% have HIV. The high percentage of patients suffering from hypertension and diabetes may be a significant contributor to the high utilization of healthcare services.

FIG. 9 is a graphical illustration of one exemplary application of the patient cluster identification system and method described herein. From an analysis of the patient clusters, it may be seen that G1 and G8 comprise 22.7% of the patient population, but account for 69.9% of all encounters. Both groups are clinically complex, have very high prevalence of hypertension and diabetes, and utilize mental-behavior healthcare three to four times more than the average Parkland Health patient. These patients also have two times higher annual cost per patient. Most of these patients are married Hispanic females in the 18-64 age range who primarily lived in a concentrated southern Dallas corridor. 87% of the patients in G1 and G8 are obese, 39% have a history of smoking, and 15% have existing chronic kidney disease diagnosis. The existing conventional practice of enrolling these patients in multiple disease-specific programs (e.g., hypertension and diabetes) resulted in significant outpatient utilization that prove to be taxing to both the healthcare system and the patients themselves. Due to the insights gained from patient clustering analysis, the novel patient cluster identification recommendation for this G1-G8 group is to design healthcare locations and programs that consolidate clinical expertise and diagnostics to meet these patients' complex needs and better manage their health. Such an integrated practice unit designed around cardio-metabolic high-risk population in the G1 and G8 clusters in addition to easy access language translation services, smoking cessation programs, and diet and nutrition programs would best serve this patient population.

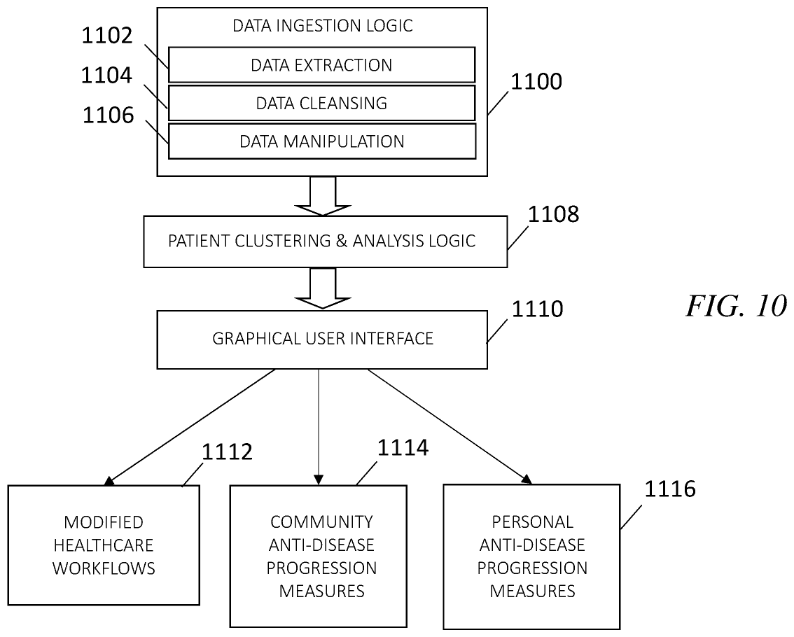

As shown in FIG. 10, a patient data ingestion logic module 1100, including logic modules that extract relevant data 1102, cleanse the data 1104, and manipulate the data 1106, receives and processes the clinical and non-clinical data associated with the patient population so that they may be analyzed by a patient clustering & analysis logic module 1108. The patient cohort to be analyzed to form patient clusters according to the teaching herein may be, for example, patients that utilize the healthcare services of a single hospital, patients of a healthcare system that includes multiple locations and virtual healthcare services (e.g., PH), or insured members of a health insurance policy or health insurance company. Alternatively, the patient cohort for analysis may be the residents of a city, county, state, region, or country, as long as the clinical and non-clinical data of this cohort are available. The data ingestion logic module 1100 may reside in the cloud or in multiple computer servers. The data ingestion logic module 1100 extracts and “cleans” or pre-processes the data, putting structured data in a standardized format and preparing unstructured text for natural language processing (NLP). This logic module 1100 may also convert the data into desired formats (e.g., text date field converted to numeric for calculation purposes). The patient clustering logic module 1108 may include artificial intelligence (AI), machine learning (ML), and NLP to analyze the ingested data. NLP is used, for example, to process raw data pulled from Dallas County Health and Human Services (DCHHS) and disease registries. A number of complex natural language processing functions including text pre-processing, lexical analysis, syntactic parsing, semantic analysis, handling multi-word expression, word sense disambiguation, and other functions may be performed. The patient clustering & analysis logic module 1108 performs the functions of classifying a patient as a member of a specific cluster identified and described above, helping to predict or anticipate the patient's needs, and generating a more tailored care plan one or more patient clusters. Additionally, the patient clustering & analysis logic module 1108 may further perform the task of analyzing data associated with the existing patient population and make recommendations on how the patient population can be clustered based on a given set of inherent data features, including demographics, utilization, and SDoH data features, and a given set of descriptive data features. The patient cluster data and analyses generated by the system and method may automatically influence and/or modify the healthcare workflows 1112, design and institute anti-disease progression measures 1114, and inform individuals so that they may formulate their own personal anti-disease progression measures 1116.

Although the patient clustering system and method described herein place emphasis on clinical access and SDoH factors, the metrics used for defining the clusters (the cluster-defining metrics) may be tweaked to highlight other types of factors to change the focus of the algorithm.

The graphical user interface (GUI) 1110 may present the data generated by the patient clustering logic module 1108 in a number of ways, including text, heatmap of patient clusters superimposed over city/county maps, detailed workflows, and patient care plans.

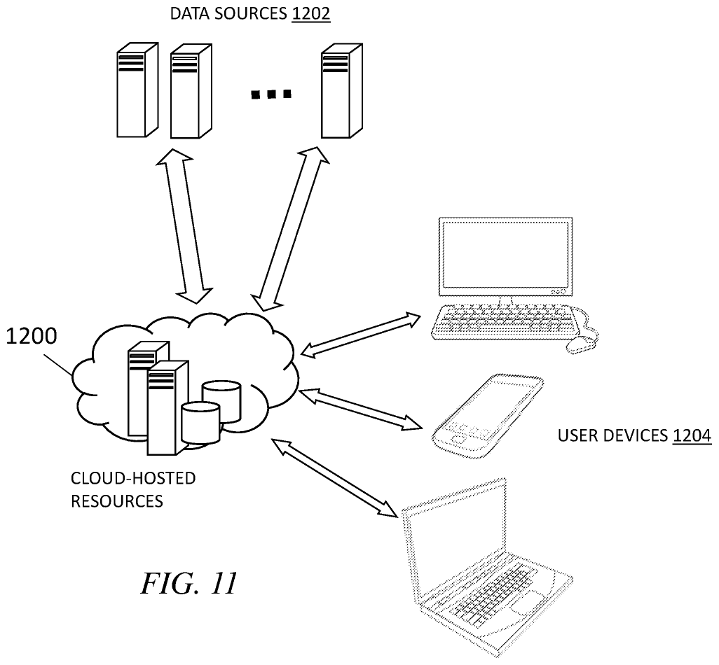

Referring to FIG. 11, The patient clustering system and method may be hosted in computing and storage resources such as, for example, on the Microsoft Azure Cloud 1200. By hosting everything on a single platform in an exemplary embodiment, the system is a streamlined process for ingesting data from disparate data sources 1202, and for cleaning, extracting, and analyzing the clinical and non-clinical data. The data presentation is performed on one or more user devices 1204 that may include mobile phones, laptops, notepad computers, desktop computers, and other user interface and display devices now known or to be developed.

Although the methodology disclosed herein cite specific numbers of features and clusters, it should be understood that the number of collected features from the data and the number of features used to define a certain number of patient clusters will vary depending on many factors, and the system and method described herein are not so limited.

The features of the present invention which are believed to be novel are set forth below with particularity in the appended claims. However, modifications, variations, and changes to the exemplary embodiments described above will be apparent to those skilled in the art, and the system and method described herein thus encompasses such modifications, variations, and changes and are not limited to the specific embodiments described herein.

What is claimed is:

1. A computerized system comprising:
a data ingestion logic module configured to automatically receive, cleanse, and process data associated with a plurality of patients to create a plurality of holistic patient records, the patient records including identity and demographic metrics associated with each patient, clinical metrics associated with each patient, clinical utilization metrics associated with each patient, social determinants of health metrics associated with each patient at the blockgroup level, and social determinants of health metrics associated with each patient at the census track level;
a first clustering logic module configured to remove multicollinear metrics from the patient records, and identify a plurality of cluster-defining metrics and a plurality of cluster-descriptor metrics, the cluster-defining metrics including demographics metrics, insurance coverage metrics, healthcare utilization metrics, and social determinants of health metrics;
a second clustering logic module configured to automatically identify a plurality of patient clusters based on the cluster-defining metrics, assign each patient to an identified patient cluster, and generate, based at least in part of the identified patient clusters, respective cluster-descriptor metrics of each patient cluster, and the patients in each patient cluster, a recommendation of at least one of: improved healthcare workflows, community-based anti-disease progression measures, personal anti-disease progression measures, an individualized and targeted care plan for a particular patient, a targeted care plan for a subset of patients, a targeted multidimensional program for patients in a patient cluster, a targeted multidimensional program for patients in multiple patient clusters; and
a graphical user interface configured to present output data associated with the recommendation based on the identified patient clusters, respective cluster-descriptor metrics of each patient cluster, and the patients assigned to the patient clusters.

2. The system of claim 1, wherein the second clustering logic module is configured to utilize Uniform Manifold Approximation and Projection (UMAP) algorithm to reduce dimensionality of the data metrics in the patient records.

3. The system of claim 1, wherein the second clustering logic module is configured to utilize HDBScan to form the clusters and assign the patient records to the clusters.

4. The system of claim 1, wherein the first clustering logic module is configured to temporarily remove medical diagnostic metrics from the patient records.

5. The system of claim 4, wherein the second clustering logic module is configured to reintroduce at least one of the removed medical diagnostic metrics into the patient records after the clusters are defined and the patient records are assigned to the clusters, where the cluster-descriptor metrics include at least one of the reintroduced medical diagnostic metrics.

6. The system of claim 1, wherein the second clustering logic module is configured to disregard medical diagnosis metrics in the patient records when defining the clusters and clustering the patient records into clusters, where the cluster-descriptor metrics include at least one of the medical diagnostic metrics.

7. The system of claim 1, wherein the second clustering logic module is configured to utilize machine learning to form the clusters and assign the patient records to the clusters.

8. The system of claim 1, wherein the graphical user interface is configured to display a geographical representation of residence locations of the patients assigned to each cluster.

9. The system of claim 1, wherein the second clustering logic module is configured to identify the plurality of patient clusters based on cluster-defining metrics selected from the group consisting of: insurance coverage metrics, encounter scheduling method metrics, encounter visit status metrics, emergency department usage metrics, outpatient visit usage metrics, hospital admission metrics, virtual encounter metrics, clinical location visit metrics, length of hospital stay metrics, oncology encounter metrics, mental health encounter metrics, women health encounter metrics, obstetrics and gynecology related encounter metrics, and social determinant of health metrics including at least one of: family structure metrics, transportation mode metrics, employment metrics, transportation access metrics, housing metrics, and education metrics.

10. A computerized method comprising:
automatically receiving data associated with a plurality of patients including identity and demographic metrics associated with each patient, clinical metrics associated with each patient, clinical utilization metrics associated with each patient, social determinants of health metrics associated with each patient at the blockgroup level, and social determinants of health metrics associated with each patient at the census track level;
automatically cleansing and processing the received data associated with a plurality of patients to create a plurality of holistic patient records;
automatically editing metrics from the patient records to remove multicollinearity among metrics;
automatically identifying a plurality of cluster-defining metrics and a plurality of cluster-descriptor metrics, the cluster-defining metrics including demographics metrics, insurance coverage metrics, healthcare utilization metrics, and social determinants of health metrics, but not including disease diagnosis metrics;
automatically identifying a plurality of patient clusters based on the cluster-defining metrics;
automatically assigning each patient to an identified patient cluster; and
generating, based at least in part of the identified patient clusters, respective cluster-descriptor metrics of each patient cluster, and the patients in each patient cluster, a recommendation of at least one of: improved healthcare workflows, community-based anti-disease progression measures, personal anti-disease progression measures, an individualized and targeted care plan for a particular patient, a targeted care plan for a subset of patients, a targeted multidimensional program for patients in a patient cluster, a targeted multidimensional program for patients in multiple patient clusters.

11. The method of claim 10, further comprising presenting, via a graphical user interface, output data associated with the recommendation based on the identified patient clusters, respective cluster-descriptor metrics of each patient cluster, and the patients assigned to the patient clusters.

12. The method of claim 10, wherein automatically identifying a plurality of cluster-defining metrics comprises utilizing Uniform Manifold Approximation and Projection (UMAP) algorithm to reduce dimensionality of the data metrics in the patient records.

13. The method of claim 10, wherein automatically identifying a plurality of cluster-defining metrics comprises utilizing HDB Scan to form the clusters and assign the patient records to the clusters.

14. The method of claim 10, wherein automatically identifying a plurality of cluster-defining metrics comprises temporarily removing medical diagnostic metrics from the patient records.

15. The method of claim 14, further comprising reintroducing at least one of the removed medical diagnostic metrics into the patient records after defining the clusters and assigning the patients to the clusters, where the cluster-descriptor metrics include at least one of the reintroduced medical diagnostic metrics.

16. The method of claim 10, further comprising disregarding medical diagnosis metrics in the patient records when defining the clusters and clustering the patient records into clusters, where the cluster-descriptor metrics include at least one of the medical diagnostic metrics.

17. The method of claim 10, further comprising utilizing machine learning to form the clusters and assign the patient records to the clusters.

18. The method of claim 10, further comprising displaying a geographical representation of residence locations of the patients assigned to each cluster.

19. The method of claim 10, wherein automatically identifying a plurality of patient clusters comprising identifying the clusters based on cluster-defining metrics selected from the group consisting of: insurance coverage metrics, encounter scheduling method metrics, encounter visit status metrics, emergency department usage metrics, outpatient visit usage metrics, hospital admission metrics, virtual encounter metrics, clinical location visit metrics, length of hospital stay metrics, oncology encounter metrics, mental health encounter metrics, women health encounter metrics, obstetrics and gynecology related encounter metrics, and social determinant of health metrics including at least one of: family structure metrics, transportation mode metrics, employment metrics, transportation access metrics, housing metrics, and education metrics.

20. A computerized system comprising:
a data ingestion logic module configured to automatically receive, cleanse, and process data associated with a plurality of patients to create a plurality of holistic patient records, the patient records including identity and demographic metrics associated with each patient, clinical metrics associated with each patient, clinical utilization metrics associated with each patient, social determinants of health metrics associated with each patient at the blockgroup level, and social determinants of health metrics associated with each patient at the census track level;
a first clustering logic module configured to remove multicollinear and medical diagnosis metrics from the patient records, and identify a plurality of cluster-defining metrics and a plurality of cluster-descriptor metrics, the cluster-defining metrics being selected from the group consisting of: insurance coverage metrics, encounter scheduling method metrics, encounter visit status metrics, emergency department usage metrics, outpatient visit usage metrics, hospital admission metrics, virtual encounter metrics, clinical location visit metrics, length of hospital stay metrics, oncology encounter metrics, mental health encounter metrics, women health encounter metrics, obstetrics and gynecology related encounter metrics, and social determinant of health metrics including at least one of: family structure metrics, transportation mode metrics, employment metrics, transportation access metrics, housing metrics, and education metrics, and the cluster-descriptor metrics including medical diagnosis metrics;
a second clustering logic module configured to automatically identify a plurality of patient clusters based on the cluster-defining metrics, assign each patient to an identified patient cluster, and generate, based at least in part of the identified patient clusters, respective cluster-descriptor metrics of each patient cluster, and the patients in each patient cluster, a recommendation of at least one of: improved healthcare workflows, community-based anti-disease progression measures, personal anti-disease progression measures, an individualized and targeted care plan for a particular patient, a targeted care plan for a subset of patients, a targeted multidimensional program for patients in a patient cluster, a targeted multidimensional program for patients in multiple patient clusters; and
a graphical user interface configured to present output data associated with a recommendation of at least one of: improved healthcare workflows, community-based anti-disease progression measures, personal anti-disease progression measures, an individualized and targeted care plan for a particular patient, a targeted care plan for a subset of patients, a targeted multidimensional program for patients in a patient cluster, a targeted multidimensional program for patients in multiple patient clusters based on the identified patient clusters, respective cluster-descriptor metrics of each patient cluster, and the patients assigned to the patient clusters.
